(19) United States
(12) Patent Application Publication
(10) Pub. No.: US 2015/0272431 A1
(43) Pub. Date: Oct. 1, 2015

(54) OBSERVATION ATTACHMENT AND DISPLAY APPARATUS

(71) Applicant: NIDEK CO., LTD., Gamagori-shi (JP)

(72) Inventors: Hironori FUJII, Toyokawa-shi (JP); Junpei NISHIYAMA, Gamagori-shi (JP); Naoki ISOGAI, Nishio-shi (JP); Masahiko KOBAYASHI, Nukata-gun (JP)

(21) Appl. No.: 14/668,338

(22) Filed: Mar. 25, 2015

(30) Foreign Application Priority Data
Mar. 31, 2014 (JP) 2014-074747

(51) Int. Cl. A61B 3/00; A61B 5/00; A61B 3/10; G02B 21/00; A61B 3/13

(52) U.S. Cl. CPC A61B 3/005; G02B 21/0012; A61B 3/13; A61B 3/102; A61B 5/0066; A61B 5/7445

(57) ABSTRACT

There is provided an observation attachment and a display apparatus achieving reduction in a burden of visual line movement of a user. The observation attachment is mounted on a surgical microscope having an eyepiece. The observation attachment includes a mounting member attached to the surgical microscope and a display holding member which holds a display provided with a display screen displayed with information for assisting a work of a user in using the surgical microscope, the information being different from an object's image directly observed through the eyepiece, and places the display screen of the display around the eyepiece of the surgical microscope in an attached state that the mounting member is attached to the surgical microscope.

### CROSS-REFERENCE TO RELATED APPLICATIONS

This application is based upon and claims the benefit of priority from the prior Japanese Patent Application No. 2014-074747 filed on Mar. 31, 2014, the entire contents of which are incorporated herein by reference.

### BACKGROUND OF THE INVENTION

1. Field of the Invention

The present disclosure relates to an observation attachment and a display apparatus.

2. Related Art

Heretofore, as one type of an observation apparatus for zooming and observing an object's tissue to be examined, a surgical microscope is known, for example. The surgical microscope is provided with at least one eyepiece. A user of the surgical microscope observes a front image of the inside of the object by looking into the eyepiece (see Patent Document 1). While the inside of the object is observed through the eyepiece, a work such as surgery, diagnosis, and others is performed.

### RELATED ART DOCUMENTS

### Patent Documents

Patent Document 1: JP-A-2005-165292

### SUMMARY OF INVENTION

### Problems to be Solved by the Invention

For more preferable work of the user using the observation apparatus, it is conceived to display information for assisting the work on a display apparatus which is separately provided from the observation apparatus. However, a screen of the conventional display apparatus has been placed apart from the observation apparatus, and therefore visual line movement between the eyepiece of the observation apparatus and the screen of the display apparatus used to be laborious for the user. The larger the burden of the visual line movement becomes, the more difficult it becomes for the user to properly perform the work.

The present disclosure has been made in view of the circumstances to solve the above problem and has a purpose to provide a observation attachment and a display apparatus achieving reduction in a burden of user's visual line movement.

### Means of Solving the Problems

To achieve the above purpose, one aspect of the disclosure provides an observation attachment mounted on an observation apparatus including an eyepiece, the observation attachment including: a connector attached to the observation apparatus; and a holder configured to hold a display provided with a display screen for displaying information for assisting a work of a user in using the observation apparatus, the information being different from an object's image directly observed through the eyepiece, and to place the display screen of the display around the eyepiece of the observation apparatus in an attached state that the connector is attached to the observation apparatus such that the display screen is retained via the connector.

A second aspect of the disclosure provides a display apparatus including: the observation attachment described above; and a display held by the holder of the observation attachment.

### Effects of the Invention

According to the present disclosure, a burden of user's visual line movement can be reduced.

### BRIEF DESCRIPTION OF THE DRAWINGS

FIG. 1 is a schematic configuration of an observation system;

FIG. 2A is a view showing a displayed state at recording on a screen of a display apparatus;

FIG. 2B is a view showing a displayed state at playback on the screen of the display apparatus;

FIG. 3 is a front view of the display apparatus;

FIG. 4 is a plan view of the display apparatus;

FIG. 5A is a view showing an attached state that a mounting member is attached to a lens barrel;

FIG. 5B is a view showing a modified example in which the mounting member is attached to a lens barrel having a diameter smaller than that of the lens barrel shown in FIG. 5A;

FIG. 5C is a view showing the mounting member of the modified example seen from a direction indicated with an arrow I in FIG. 5A;

FIG. 6 is a schematic view of a mounting mechanism according to a modified example; and

FIG. 7 is a schematic view of a mounting mechanism according to a modified example.

### DESCRIPTION OF EMBODIMENTS

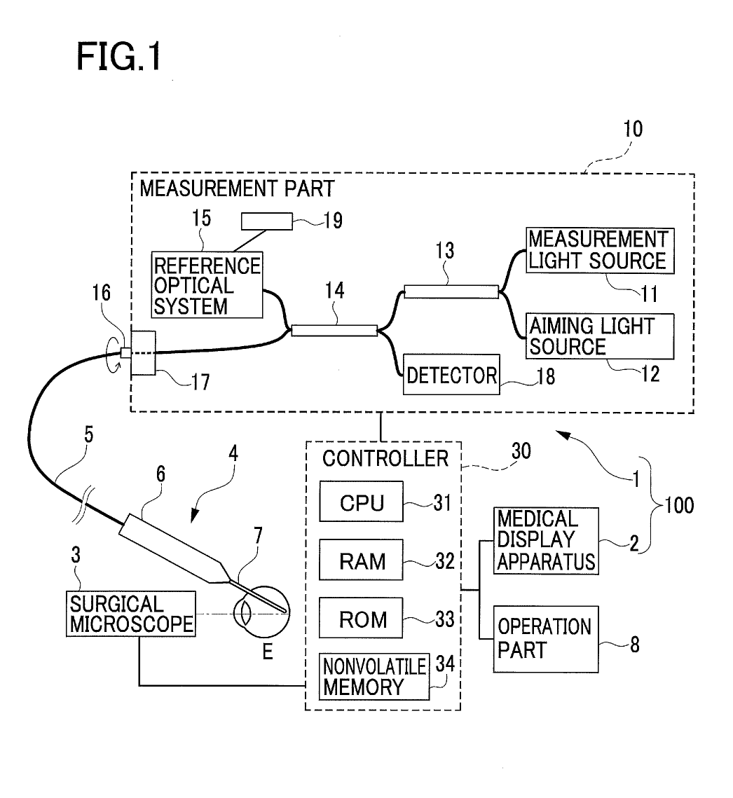

A detailed description of a preferred embodiment of the present disclosure is now given with reference to the accompanying drawings. Firstly, with reference to FIG. 1, a schematic configuration of an observation system 100 according to the present embodiment is explained. The observation system 100 of the present embodiment mainly includes an optical coherence tomography device (OCT device) 1 and a display apparatus 2. Further, the observation system 100 may also include an observation apparatus such as a surgical microscope 3.

The optical coherence tomography device (OCT device) 1 photographs a tomographic image of a tissue inside an object to be examined by use of a probe 4 inserted in the object. In the present embodiment, explanation is made with illustrating an ophthalmic imaging apparatus configured to photograph a tomographic image of an inner tissue (for example, a retina) of an examinee's eye E, but the present disclosure can also be applied to an apparatus for photographing a tomographic image of an object other than eyes (for example, internal organs, ears, and others). The OCT device 1 includes a measurement part 10 and a controller 30.

The measurement part 10 is provided with a configuration of an optical coherence tomography (OCT) (for example, a coherence optical system). The measurement part 10 of the present embodiment includes a measurement light source 11, an aiming light source 12, a coupler 13, a coupler 14, a reference optical system 15, a mounting part 16, a fiber rotating motor 17, a detector (light receiving element) 18, and an optical path length changing unit 19.

The measurement light source 11 emits light for obtaining a tomographic image. As one example, the OCT device 1 of the present embodiment is provided with the measurement light source 11 which is able to change a wavelength of emitted laser beam at high speed so that the tomographic image is obtained by Swept-source OCT (SS-OCT) measurement. The measurement light source 11 of the present embodiment includes a laser medium, a resonator, a wavelength selectable filter, and others. As the wavelength selectable filter, for example, a combination of diffraction grating and polygon mirror or a filter using Fabry-Perot etalon can be adopted.

The aiming light source 12 emits aiming light as visible light for indicating an irradiation point of measurement light to be photographed (namely, a photographed position of the tomographic image).

The coupler 13 is configured to synthesize the light emitted from the measurement light source 11 and the aiming light emitted from the aiming light source 12 so that two optical axes of the lights coincide each other. The coupler 14 splits the light emitted from the coupler 13 into measurement light (sample light) and reference light. The measurement light is introduced to the probe 4 which is attached to the mounting part 16. The reference light is introduced to the reference optical system 15. Moreover, the coupler 14 synthesizes the measurement light (reflection measurement light) reflected on the examinee's eye E with the reference light produced by the reference optical system 15 so as to produce coherence light. The coupler 14 makes the detector 18 to receive the produced coherence light.

The reference optical system 15 is configured to return the reference light introduced from the coupler 14 to the coupler 14 again. The reference optical system 15 may be a Michelson optical system or a Mach-Zehnder optical system. In the present embodiment, the reference optical system 15 includes a reflective optical system provided with a reference mirror and others that reflects the reference light introduced from the coupler 14 and return the reference light to the coupler 14 again. As mentioned above, the reference light returned to the coupler 14 is synthesized with the reflection measurement light reflected on the examinee's eye E. The configuration of the reference optical system 15 may be modified. For instance, the reference optical system 15 may be configured to transmit the reference light introduced from the coupler 14 to the detector 18 by a transmission optical system such as an optical fiber without reflecting the reference light.

To the mounting part (for example, a connector) 16, a rear end portion (base end portion) of a fiber 5 of the probe 4 is attached in a detachable manner. The probe 4 of the present embodiment is provided with the fiber 5, a handpiece 6, and a needle 7. The fiber 5 is configured to introduce the measurement light introduced from the coupler 14 of the measurement part 10 and the aiming light to a distal end portion of the needle 7. The fiber 5 covered with torque coil (not shown) is rotatable with respect to the handpiece 6. The handpiece 6 is an almost cylindrical member to be held by a user (for example, an examiner, an operator, and others) of the observation apparatus. The needle 7 formed on a distal end of the handpiece 6 has an outer diameter smaller than an outer diameter of the handpiece 6. The distal end portion of the needle 7 is to be inserted into the object to be examined (for example, the examinee's eye E). The fiber 5 is connected to a rear end portion of the handpiece 6 and extends to the distal end portion of the needle 7. The probe 4 is configured to emit the measurement light and the aiming light, which is introduced therein through the fiber 5, from a distal end portion of the probe 4 while scanning the lights. A detailed structure of the distal end portion of the probe 4 is omitted with its explanation.

The fiber rotating motor 17 is configured to rotate the mounting part 16 attached with the fiber 5 of the probe 4 about an axis of the fiber 5. Specifically, the fiber rotating motor 17 rotates the mounting part 16 to rotate the fiber 5 so that the measurement light and the aiming light are scanned.

The detector 18 is configured to detect a coherence state of the reflection measurement light and the reference light. In other words, the detector 18 is configured to detect a coherence signal of the coherence light produced by the coupler 14. To be specific, in a case of Fourier-domain OCT, a spectral intensity of the coherence light is detected by the detector 18 and a depth profile (A scan signal) in a predetermined range is obtained by performing Fourier transform of spectral intensity data. As mentioned above, the SS-OCT is adopted as the OCT device 1 of the present embodiment, but alternatively, various OCT may be applied as the OCT device 1. For example, either one of Spectral-domain OCT (SD-OCT), Time-domain OCT (TD-OCT) and others may be adopted as the OCT device 1. When the SS-OCT is adopted, it is preferable to adopt a balance detector having a plurality of light receiving elements as the detector 18. When the balance detector is used, the OCT device 1 obtains the difference among coherence signals from the plurality of the light receiving elements, and thereby unnecessary noise included in the coherence signals can be reduced. As a result, a quality of the tomographic image is improved.

The measurement part 10 includes the optical path length changing unit 19 that changes the difference in optical path length of the measurement light and the reference light. The optical path length changing unit 19 of the present embodiment includes an optical member (for example, a reference mirror) provided in the reference optical system 15 and a drive part. The reference mirror is placed in an optical path of the reference light and moved in an optical axial direction of the reference light by the drive part. As a result, the difference of the optical path length between the measurement light and the reference light is changed. Herein, a configuration for changing the difference of the optical path length may be placed on an optical path of the measurement light. The OCT device 1 further includes various configurations such as an optical system for performing focus adjustment of the measurement light, but a detailed explanation of those is omitted.

The controller 30 is provided with a CPU (processor) 31, an RAM 32, an ROM 33, a nonvolatile memory 34, and others. The CPU 31 is in charge of controlling the OCT device 1 and peripheral devices. The RAM 32 is configured to temporarily store various information. The ROM 33 stores various programs, an initial value, and others. The nonvolatile memory 34 is a non-fugitive storage medium which can retain stored contents even when power supply is shut off. For example, a hard disk drive, a flash ROM, a USB memory stick attached to the OCT device 1 in a detachable manner, and others may be used as the nonvolatile memory 34. In the nonvolatile memory 34, various information such as a photographed tomographic image is stored.

To the controller 30, peripheral devices such as the display apparatus 2, the surgical microscope 3, and an operation part 8 are electrically connected.

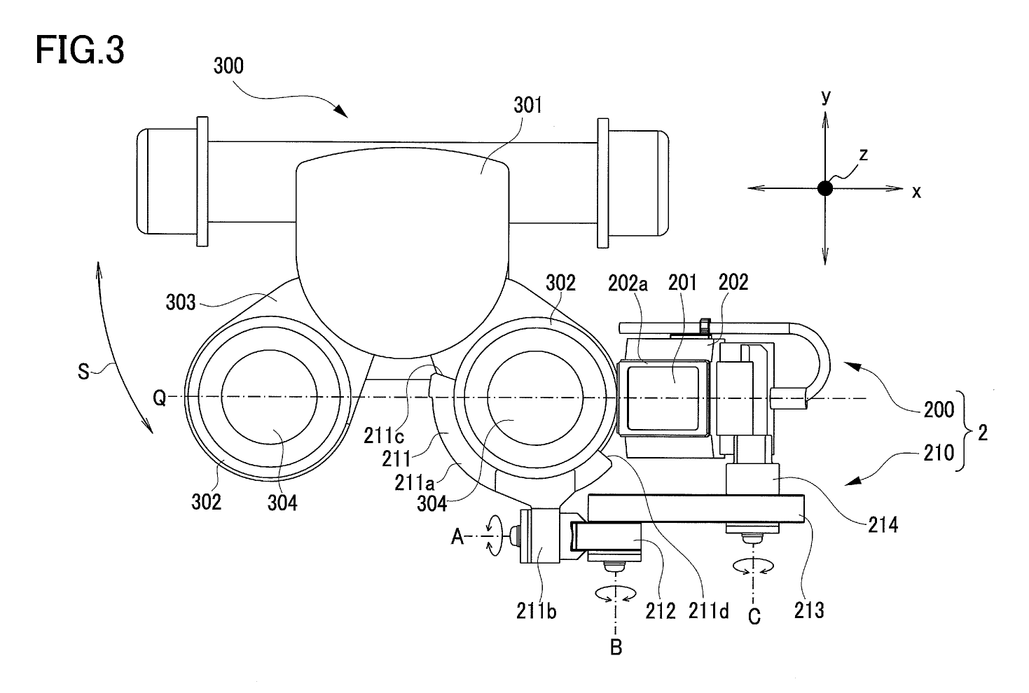

The surgical microscope 3 is used for observing a front image of the object to be examined (in the present embodiment, the examinee's eye E). The surgical microscope 3 is configured to zoom in (enlarge) and display (to photograph, enlarge, and display in the present embodiment) the inside of the object (the examinee's eye E in the present embodiment). A user (for example, an examiner, an operator, and others) of the surgical microscope 3 looks into eyepieces 304 (see FIG. 3) of the surgical microscope 3 to observe the inside of the object when performing surgery, diagnosis, and training for these surgery and diagnosis (they are called as “work” in the present embodiment).

The display apparatus 2 is a device for displaying information for assisting the work which is performed with observing the object by an observation apparatus (the surgical microscope 3 in the present embodiment). The information displayed by the display apparatus 2 includes at least either one of image information such as a still image or a moving image and text information such as letters and characters. The information for assisting the work may be the one obtained based on the actual object or the one prepared in advance. As the information for assisting the work, for example, photographed image data of the object (to be specific, tomographic image data, fluorescent image data, enlarged photographed image data, and others), clinical chart data of the object, manual data such as a surgery method, observation condition data of the observation apparatus, and others are exemplified.

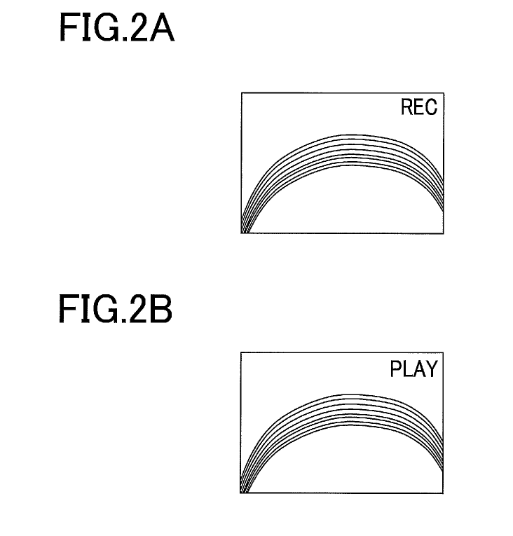

In the present embodiment, the display apparatus 2 displays a tomographic image photographed by the OCT device 1, which is a different type of apparatus (different in modality) from the surgical microscope 3. In the present embodiment, a display of the tomographic image is controlled by the controller 30. The controller 30 controls the display apparatus 2 to display a live image of the tomographic image of the object's tissue (see FIG. 2A) that is photographed in real time by the OCT device 1 in the present embodiment. In a surgery (for example, a vitreous humor surgery), there may be a case that an incision of the object in which the probe 4 is inserted is also used for inserting a surgical instrument into the object. In this case, the surgical instrument is inserted after the probe 4 is taken out from the object. Namely, during the surgery, there is a case that the tomographic image cannot be photographed by the probe 4. In response to this, the observation system 100 of the present embodiment is configured to display the past tomographic image (for example, past live image) recorded (or obtained) in advance by the OCT device 1 on the display apparatus 2.

The image photographed by the OCT device 1 is recorded by the controller 30. This recorded image is stored in the nonvolatile memory 34. In the present embodiment, the controller 30 can record the live image in parallel with displaying the image by the display apparatus 2. While recording is performed, the controller 30 controls the display apparatus 2 to indicate the fact of recording on a screen with the tomographic image. As shown in FIG. 2A, for example, a text of “REC,” which means recording is ongoing, is combined with the tomographic image of the object's tissue and displayed. Thus, the user can understand the displayed image is being recorded.

Further, the controller 30 controls the display apparatus 2 to display (play back) the image stored (recorded) in the nonvolatile memory 34 when the controller 30 receives a command from the user and others. In the present embodiment, the display apparatus 2 continuously displays the past live image (like moving image). While the images are replayed, the controller 30 controls the display apparatus 2 to indicate the fact of replaying on the screen. For example, as shown in FIG. 2B, a text of “PLAY,” which means the images are played back, is combined with the tomographic image of the object's tissue and displayed. As a result, the user can understand that the recorded images are being replayed. The recorded images and the images displayed during the playback are not necessarily the live images and may be still images.

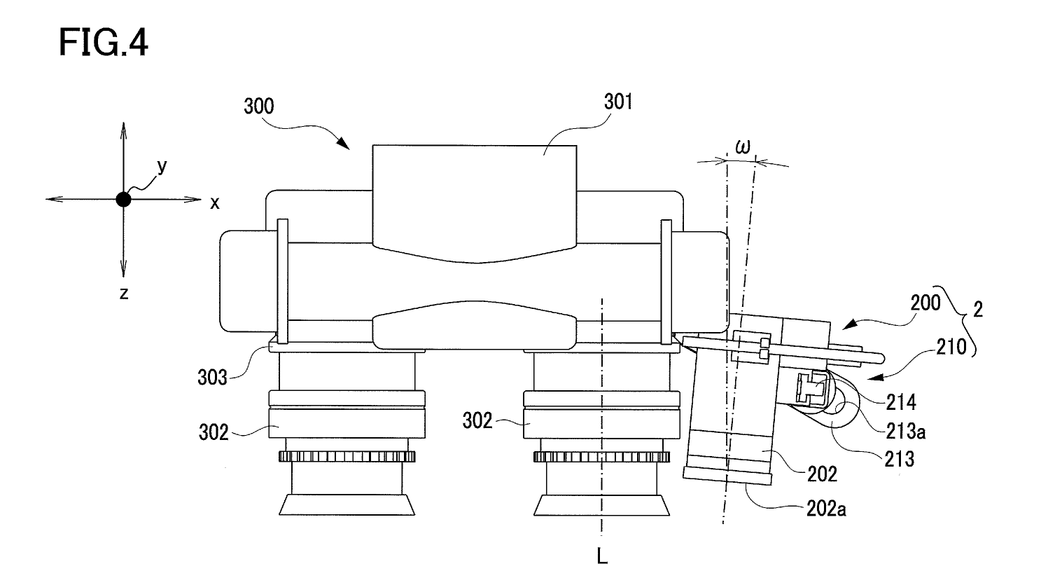

In the present embodiment, the display apparatus 2 is attached to the surgical microscope 3 and then used, which will be explained in detail below. A detailed configuration of the display apparatus 2 will be explained below with reference to FIGS. 3 and 4. Herein, in addition to the display apparatus 2, the OCT device 1 may be connected to another display apparatus which is separately provided from the display apparatus 2. This separately provided display apparatus may be a similar one to the display apparatus 2 and may be an apparatus for general purpose such as a monitor for a personal computer.

The operation part 8 is a device for recognizing various operation commands from the user. As the operation part 8, for example, at least one of a mouse, a joy stick, a touch screen, or others may be adopted.

With reference to FIGS. 3 and 4, a detailed configuration of a lens barrel unit 300 provided with the eyepieces 304 in the surgical microscope 3 and the display apparatus 2 is now explained. In FIGS. 3 and 4, each of a left and right direction, an upper and lower direction, and a forward and backward direction of the lens barrel unit 300 is respectively indicated with an arrow x, an arrow y, and an arrow z.

In the present embodiment, the lens barrel unit 300 includes a base part 301, lens barrels 302, a lens barrel holding part 303, and the eyepieces 304. One or a plurality of the lens barrel units 300 may be provided in one surgical microscope 3.

The lens barrel 302 holds the eyepiece 304. The lens barrel 302 is configured to change a position of the eyepiece 304 in a predetermined range in a lens axis L direction of the eyepiece 304 (see FIG. 4). In the present embodiment, two lens barrels 302 are provided in one-by-one correspondence with both eyes of the user. The lens barrel holding part 303 is configured to open and close in a direction indicated with an arrow S (see FIG. 3) with respect to the base part 301 so that an interval between the left and right lens barrels 302 is changed. In observation, the interval of the left and right lens barrels 302 is adjusted to correspond to an interval of the user's both eyes. An image of the inside of the object (a front image of the examinee's eye in the present embodiment) is zoomed in by a photographing optical system (not shown) of the surgical microscope 3, and then the eyepieces 304 project this image on the user's eyes (not shown) that look into the eyepieces 304 (and the lens barrels 302). In the present embodiment, the eyepiece 304 is located in a deeper position in the lens barrel 302 than an opening thereof.

In the present embodiment, the display apparatus 2 mainly includes a display 200 and a mounting mechanism 210. In the present embodiment, the display 200 includes a monitor structure for displaying the tomographic image of the tissue of the examinee's eye E obtained by the OCT device 1. The display 200 has a display screen 201 and a housing 202. In the present embodiment, the housing 202 is formed on a front side (a front side on a paper of FIG. 3) with an observation window 202a and formed in a cylindrical shape. In the present embodiment, a transparent plate (a flat plate made of glass or plastic, a lens, and so on) is put in place in the observation window 202a of the display 200, and the display screen 201 (for example, a liquid crystal panel) is in a position located behind and spaced from the observation window 202a (i.e., transparent plate). In the present embodiment, the display screen 201 is placed in a position so as to be visually recognizable for the user in looking into the observation window 202a.

The mounting mechanism 210 corresponds to an observation attachment attached (mounted) to an observation apparatus having an eyepiece (in the present embodiment, the surgical microscope 3). As well as holding the display 200, the mounting mechanism 210 is attached to the surgical microscope 3 and arranged so that the display screen 201 is placed around the eyepiece 304 of the surgical microscope 3 while the mounting mechanism 210 is in an attached state to the surgical microscope 3. In the attached state, for example, the display screen 201 is arranged side by side with the eyepiece 304 on any one of upper, lower, left, and right sides of the eyepiece 304. Alternatively, the display screen 201 may be placed obliquely (upper-right, upper-left, lower-right, or lower-left position) relative to the eyepiece 304. Further, as shown in FIGS. 3 and 4, the mounting mechanism 210 is allowed to be placed such that a front side of the display screen 201 faces to the user simultaneously with a rear side of the eyepiece 304 (namely, a side closer to an opening of the lens barrel 302). The mounting mechanism 210 thus arranges the display screen 201 around the eyepiece 304, and thereby the user can successively observes the front image of the inside of the object directly observed through the eyepieces 304 of the surgical microscope 3 and the tomographic image of the object's tissue photographed by the OCT device 1 with less visual line movement. Further, as shown in FIGS. 3 and 4, the mounting mechanism 210 of the present embodiment arranges the display screen 201 on either one of the left or right side of the lens barrel 302. To be concrete, the mounting mechanism 210 of the present embodiment arranges the display screen 201 outside the left and right eyepieces 304 with respect to the surgical microscope 3 for binocular observation. It is easier to create an installation space outside than inside (between) the left and right eyepieces 304, and therefore the display apparatus 2 is preferably attached to the surgical microscope 3 for binocular observation.

In the example shown in FIGS. 3 and 4, vertical and lateral dimensions of the display screen 201 are almost same as a diameter (effective diameter) of the eyepiece 304. Thereby, when the user's visual line moves between the eyepieces 304 and the display screen 201, the user can observe the object with a similar angle of view without moving the user's eye forward or backward. Therefore, the user can smoothly perform the observation.

Further, as mentioned above, the image of the inside of the object obtained by the surgical microscope 3 and the tomographic image photographed by the OCT device 1 are respectively observed by looking into the lens barrels 302 and the observation window 202a. Accordingly, when the user moves his/her visual line between the eyepieces 304 and the display screen 201, the user is unlikely to have uncomfortable feeling.

As shown in FIG. 3, in the present embodiment, the mounting mechanism 210 includes a mounting member (connector) 211, a link fixing member 212, a link member 213, and a display holding member (holder) 214.

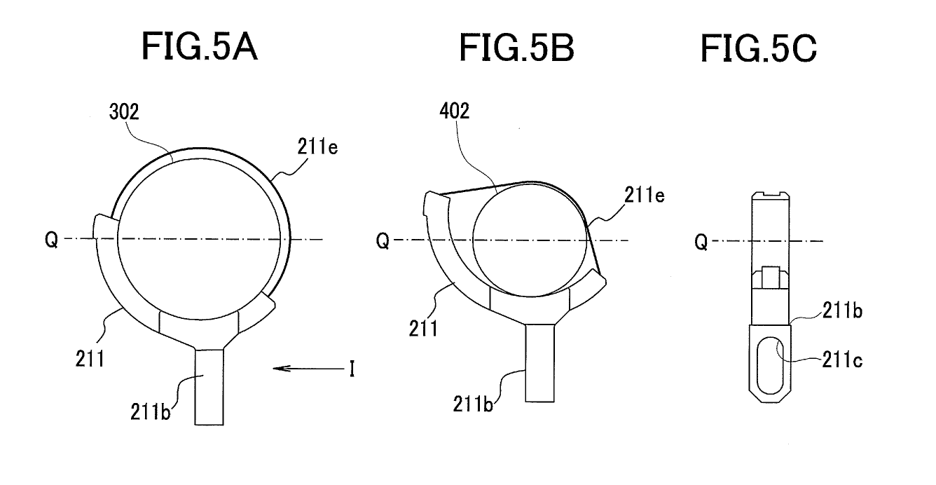

The mounting member 211 is used for fixing the display apparatus 2 to the surgical microscope 3. As shown in FIG. 3, the mounting member 211 is formed with a body part 211a and a connection part 211b. The body part 211a is formed in an arcuate shape, and an arcuate inner surface of the body part 211a is brought into contact with the lens barrel 302. In the example shown in FIG. 3, the arcuate inner surface is formed along a curved shape of the lens barrel 302. On both ends 211c and 211d of the body part 211a, a band 211e (for example, a fixing band) is connected (see FIG. 5A). By winding the band around the lens barrel 302, the mounting member 211 holds the lens barrel 302 along an outer circumference of the lens barrel 302. As a result, the mounting member 211 is fixed to the lens barrel 302. As mentioned above, in the present embodiment, the mounting member 211 is mounted along the outer circumference of the lens barrel 302, so that the display apparatus 2 is fixed to the surgical microscope 3. The mounting member 211 can be attached in any position along the curved shape of the outer circumference of the lens barrel 302.

To the connection part 211b, the link fixing member 212 is connected. The connection part 211b is formed as radially protruding from an arcuate outer circumferential portion of the body part 211a. The connection part 211b has a through hole (not shown) formed in a direction same with a direction in which the body part 211a extends (the direction x). The link fixing member 212 is inserted in the through hole to be connected to the mounting member 211. Herein, the link fixing member 212 is retained in a rotatable manner to rotate about an axis A extending in a depth direction of the through hole.

The link fixing member 212 is connected its one end with the link member 213 so that the link member 213 is allowed to rotate or pivot about an axis B intersecting the axis A (in the present embodiment, the axis B is orthogonally intersects the axis A).

The link member 213 is formed with a long hole (slit) 213a penetrating in a direction same with the axis B, the long hole 213a extending in a longitudinal direction of the link member 213 (see FIG. 4). The display holding member 214 is retained by the long hole 213a. In the present embodiment, the link member 213 supports the display holding member 214 in this manner. The link member 213 is formed with a slide mechanism (hereinafter referred as a slider) for displacing the display holding member 214 in a longitudinal direction of the link member 213 in the long hole 213a. Since the display holding member 214 supports the display 200, the display 200 can be displaced in the longitudinal direction of the link member 213 by use of the slider.

Further, the display holding member 214 is supported in a rotatable manner rotating about an axis C orthogonally intersecting a longitudinal axis of the link member 213. Thereby, the front surface of the display 200 can be adjusted in an arbitrary orientation orthogonally intersecting the axis C. In the present embodiment, the axes B and C are arranged in parallel. Herein, the display holding member 214 may be provided in a detachable manner to the display 200.

In the example shown in FIGS. 3 and 4, the mounting member 211 is arranged so that the axis A is in parallel with a center axis Q horizontally extending on a center of an upper and lower direction of the eyepiece 304. Further, a rotation angle of the link fixing member 212 relative to the mounting member 211 is determined so that the link member 213 is supported on the link fixing member 212 and that the axes B and C respectively and orthogonally intersect the lens axis L and the center axis Q of the eyepiece 304. In this manner, as shown in FIG. 3, in the attached state that the mounting member 211 is attached to the surgical microscope 3, the display screen 201 is placed around the eyepiece 304 by the display holding member 214. To be specific, the display screen 201 is placed side by side with the eyepiece 304 at the same location (on the same height or level) in the upper and lower direction. Accordingly, the user's visual line movement between the eyepiece 304 and the display screen 211 becomes easier.

In this example of the present embodiment, rotation of the link member 213 about the axis B and movement of the display holding member 214 by the slider enable the position of the display 200 to be adjusted in accordance with the user's eye in a forward and backward direction and in a left and right direction within a predetermined adjustable range on a horizontal surface relative to the lens axis L of the eyepiece 304. The display apparatus 2 is, for example, able to adjust the position of the display screen 201 relative to the eyepiece 304 in the lens axis L direction by changing the position of the display 200 in the lens axis L direction of the eyepiece 304. When a difference in accommodation power of the user's eye is large between a case of looking into the eyepieces 304 and a case of looking at the display screen 201, the user's burden of the visual line movement becomes large. To reduce such a burden, in the present embodiment, the position of the display screen 201 relative to the eyepiece 304 in the lens axis L direction is adjustable. By adjusting the position of the display screen 201 in the lens axis L direction, the difference in visibility of the user's eye can be corrected. In other words, the difference in the range of accommodation in each case of looking into the eyepieces 304 and looking at the display screen 201 can be reduced in accordance with the user's visibility.

Moreover, when the display holding member 214 rotates (rotates on its axis) with respect to the link member 213, the display 200 is adjusted its angle co between the lens axis L of the eyepiece 304 and a front surface orientation of the display screen 201 (see FIG. 4). In the present embodiment, the front surface orientation of the display screen 201 can be tilted to face upward or downward relative to the lens axis L by the rotation of the link fixing member 212 about the axis A. By adjusting the front surface orientation of the display screen 201 relative to the user's eye, it becomes possible to tilt the front surface of the display screen 201 in a preferable angle for the user to see.

FIGS. 3 and 4 show a case when the mounting mechanism 210 is attached to the lens barrel 302 for the right eye. As an alternative, the mounting mechanism 210 of the present embodiment with the identical configuration may be attached to the lens barrel 302 for the left eye. When attaching to the lens barrel 302 for the left eye, the display apparatus 2 shown in FIGS. 3 and 4 is rotated by 180 degrees about the axis z. As a result, in the present embodiment, the display screen 201 is also mounted with rotated by 180 degrees. In this case, the controller 30 may be configured to control a rotation angle of an image displayed on the display screen 201. Specifically, an image rotated by 180 degrees about the axis z from the image displayed when the mounting mechanism 210 is attached to the lens barrel 302 for the right eye (top and bottom sides and left and right sides of the image are respectively turned round) may be displayed on the display screen 201 of the display apparatus 2 attached to the lens barrel 302 for the left eye. In the present embodiment, each element included in the mounting mechanism 210 is formed in a symmetrical shape so that the mounting mechanism 210 may be attached to either one of the lens barrels 302 on a left or right side.

The above explanation has been made with the embodiment, but the present disclosure is not limited to the above embodiment and may be implemented with variously modified embodiments.

For instance, in the above embodiment, the explanation is made with a configuration that the display apparatus 2 is attached to the surgical microscope 3. However, an observation apparatus to which the display apparatus 2 is attached is not limited to this. As an alternative, the display apparatus 2 may be attached to another observation apparatus having a lens barrel. For example, a slit lamp, an endoscope, a non-surgical microscope, and others may be exemplified.

In the above embodiment, the display apparatus 2 is fixed to the surgical microscope 3 by use of frictional force and others between the lens barrel 302 and the mounting member 211 formed with the band, but alternatively, in the mounting mechanism 210, the display apparatus 2 may be fixed to the surgical microscope 3 by a different means from the above embodiment. For example, each of the mounting mechanism 210 and the surgical microscope 3 may be formed with an engaging portion so that their engaging portions are engaged with each other to fix the display apparatus 2. Alternatively, the display apparatus 2 may be fixed by use of an adhesive or a magnet.

In the above embodiment, the mounting mechanism 210 is attached to the lens barrel 302, but alternatively, the mounting mechanism 210 may be attached to a position other than the lens barrel 302 of the surgical microscope 3. For example, as long as being attached to the lens barrel unit 300, the mounting mechanism 210 may be attached to either one of the base part 301 or the lens barrel holding part 303. Furthermore, the mounting mechanism 210 may be attached to a position other than the lens barrel unit 300 in a housing of the surgical microscope 3 (for example, to a stand or an arm, and others).

The mounting mechanism 210 may include a mechanism of displacing the display 200 in an upper and lower direction. For example, as shown in FIG. 5C, a through hole formed in the connection part 211b of the mounting member 211 may be a long hole (slot) 211c extending in the upper and lower direction. By changing the position where the link fixing member 212 is fixed in the long hole 211c in the upper and lower direction, the position of the display 200 is adjusted in the upper and lower direction with respect to the user's eye. For example, when the mounting mechanism 210 of the above embodiment is attached to a lens barrel 402 (see FIG. 5B) having an outer circumferential diameter different from the lens barrel 302 of the above embodiment (see FIG. 5A), the position of the display 200 is misaligned upwardly or downwardly according to the diameter of the lens barrel. In response to this, in a case that the mounting mechanism 210 is provided with a mechanism that is able to displace the display 200 in the upper and lower direction, the display 200 is displaced in position in the upper and lower direction, thereby reducing a gap between a center of the height of the eyepiece 304 and a center of the height of the display screen 201.

In the above embodiment, the link member 213 is allowed to rotate about the axis B and the slider allows the display holding member 214 to move in the longitudinal direction of the link member 213, and thereby, the display 200 is allowed to move in a predetermined range centered about an axis orthogonally intersecting the lens axis L of the eyepiece 304 and the center axis Q in the upper and lower direction of the eyepiece 304. However, the configuration for positioning the display 200 is not necessarily limited to the above configuration. Alternatively, the mounting mechanism 210 may include a link mechanism in which two or more link members are connected in series. Further, the mounting mechanism 210 may be provided with two or more sliders (for example, an x slider and a z slider) which are configured to respectively move the display 200 in different directions.

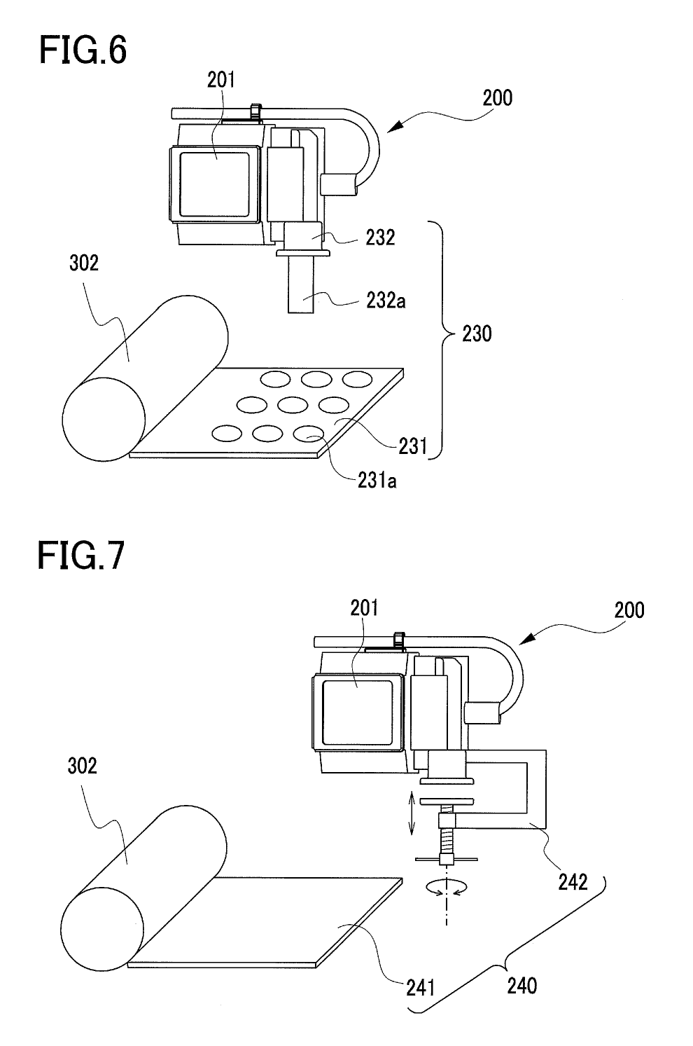

Further as shown in FIG. 6, the display 200 may be positioned by a mounting mechanism 230 including a plate-like member 231 mounted in a side face of the lens barrel 302 and a display holding member 232. In an example shown in FIG. 6, the plate-like member 231 is formed with a plurality of openings 231a, each of which has the same size and arranged on a plate surface. The display holding member 232 is formed with an insertion part 232a to be inserted in either one of the openings 231a. The insertion part 232a is inserted in either one of the openings 231a, and thereby the display 200 is placed in position. In an example shown in FIG. 7, the display 200 is placed in position by a mounting mechanism 240 including a plate-like member 241 with no openings and a display holding member 242. The display holding member 242 is provided with a vice (or a clip) for holding the plate-like member 241. In the example shown in FIG. 7, the display 200 is positioned according to a position and an orientation in which the plate-like member is fixed with the vice.

In the above embodiment, the controller 30 of the OCT device 1 is configured to adjust an orientation (angle) of the image displayed on the display screen 201, but alternatively, the controller 30 may have a configuration of controlling the image displayed on the display screen 201 to be bilaterally symmetrical or vertically symmetrical.

In the above embodiment, the mounting mechanism 210 is configured to adjust the position of the display 200 in the attached state that the mounting mechanism 210 is attached to the surgical microscope 3 within the predetermined adjustable range. The configuration is not limited to this. The mounting mechanism 210 may have the configuration with no liberty of adjusting the position of the display 200 in the attached state that the mechanism 210 is attached to the surgical microscope 3.

The above embodiment is explained with the configuration that the display 200 displays the tomographic image of the object's tissue (in the above embodiment, the tomographic image of the fundus of the examinee's eye) obtained by the OCT device 1, but when displaying the image of the object, the image displayed on the display 200 is not limited to the above-mentioned image. Other than the tomographic image of the object's tissue, the display 200 may display an image of the tissue inside the object which is different from the front image observed through the eyepieces 304 of the surgical microscope 3. For example, the display 200 displays an image different from the front image observed through the eyepieces 304 in at least one of a display mode (for instance, display magnification and others), a photographing method, and an apparatus used for photographing. To be specific, the display 200 may display an image such as an enlarged image of the image observed through the eyepieces 304 of the surgical microscope 3, a fluorescein photographed image of the inside of the object which is photographed in advance, and the object's image photographed in advance by a different type of apparatus from the surgical microscope 3.

What is claimed is:

1. An observation attachment mounted on an observation apparatus including an eyepiece, the observation attachment including:
a connector attached to the observation apparatus; and
a holder configured to hold a display provided with a display screen for displaying information for assisting a work of a user in using the observation apparatus, the information being different from an object's image directly observed through the eyepiece, and to place the display screen of the display around the eyepiece of the observation apparatus in an attached state that the connector is attached to the observation apparatus such that the display screen is retained via the connector.

2. The observation attachment according to claim 1, wherein the connector is attached to the observation apparatus by retaining a lens barrel, which holds the eyepiece, along an outer circumference of the lens barrel.

3. The observation attachment according to claim 1 includes an adjusting mechanism allowed to adjust a position of the display in the attached state.

4. The observation attachment according to claim 3, wherein the adjusting mechanism includes a forward and backward position adjuster which changes a position of the display in a lens axial direction of the eyepiece to adjust a position of the display screen in the lens axial direction with respect to a user's eye.

5. The observation attachment according to claim 3, wherein the adjusting mechanism includes a left and right position adjuster which changes a position of the display in a left and right direction of the eyepiece to adjust the position of the display screen in the left and right direction with respect to a user's eye.

6. The observation attachment according to claim 3, wherein the adjusting mechanism includes a front orientation adjuster which is configured to rotate the display about an axis intersecting a lens axis of the eyepiece to adjust an orientation of a front direction of the display screen with respect to the user's eye.

7. The observation attachment according to claim 3, wherein the adjusting mechanism includes an upper and lower position adjuster configured to adjust a height of the display screen with respect to the user's eye.

8. A display apparatus including: the observation attachment according to claim 1; and a display held by the holder of the observation attachment.

9. The display apparatus according to claim 8, wherein
the display includes a cylindrical housing formed on a front side with an observation window, and
a display screen of the display is in a position located behind and spaced from the observation window and placed to be visually recognizable for a user by looking into the housing from the observation window.

10. The display apparatus according to claim 8, wherein the display displays a tomographic image of an object's tissue on the display screen in a manner that a coherence optical system provided in an optical coherence tomography device detects a coherence signal of measurement light reflected on the object's tissue and reference light by use of a detector and the optical coherence tomography device processes the coherence signal from the detector to photograph the tomographic image.

11. The display apparatus according to claim 10, wherein the display displays the tomographic image photographed by the optical coherence tomography device on the display screen by irradiating the measurement light on the object's tissue through a probe which is inserted inside the object.
